(19) United States
(12) Patent Application Publication
(10) Pub. No.: US 2016/0249863 A1
(43) Pub. Date: Sep. 1, 2016

(54) HEALTH CONDITION DETERMINATION METHOD AND HEALTH CONDITION DETERMINATION SYSTEM

(71) Applicant: FUJITSU LIMITED, Kawasaki-shi (JP)

(72) Inventors: Takahisa ANDO, Kawasaki (JP); Seishi OKAMOTO, Hachioji (JP)

(21) Appl. No.: 15/152,625

(22) Filed: May 12, 2016

Related U.S. Application Data
(63) Continuation of application No. 13/904,136, filed on May 29, 2013.

(30) Foreign Application Priority Data
Jun. 4, 2012 (JP) 2012-126997

(51) Int. Cl. A61B 5/00; G06F 19/00; A61B 5/0205

(52) U.S. Cl. CPC A61B 5/7275; A61B 5/0205; G06F 19/345

(57) ABSTRACT

A health condition determination method including: acquiring a boundary range including one or more boundary values and a width in examination data, the examination data corresponding to an examination item in which a normal range and an abnormal range are set; identifying a plurality of determination candidate models each including a pattern for setting the normal range and the abnormal range for the examination item; calculating an accuracy corresponding to each of the plurality of determination candidate models based on model construction data corresponding to a disease related to the examination item; and determining, from the plurality of determination candidate models, a determination model that outputs whether determination data with respect to the examination item is normal or not based on the calculated accuracy.

### CROSS-REFERENCE TO RELATED APPLICATION

This application is a continuation of U.S. Ser. No. 13/904,136, filed May 29, 2013, which is based upon and claims the benefit of priority of the prior Japanese Patent Application No. 2012-126997, filed on Jun. 4, 2012, the entire contents of which are incorporated herein by reference.

### FIELD

The embodiment discussed herein is related to a health condition determination method and a health condition determination device.

### BACKGROUND

Health care guidance is provided to avoid lifestyle-related diseases. A health instructor determines a possibility that a subject will have a lifestyle-related disease in the future based on examination data such as a result of a physical examination of a subject and experiences and provides the subject with health care guidance as appropriate. Examination data such as a result of a physical examination includes numerical values of a plurality of items on a body of a subject. A boundary value for determining normal/abnormal is set for each item. It is hard to determine whether or not examination data of a subject related to examination items is normal. Deterioration of a health condition is determined based on such detection that data of a subject, which has had a normal value, is close to or exceeds over a boundary value. There is a limit on determination of a health condition which is performed by a health instructor and at the same time, manpower for health instruction has been deficient.

The related art is disclosed in Japanese Laid-open Patent Publication No. 2004-305674 or Japanese Laid-open Patent Publication No. 2009-110279.

### SUMMARY

According to one aspect of the embodiments, a health condition determination method, includes: acquiring a boundary range including one or more boundary values and a width in examination data, the examination data corresponding to an examination item in which a normal range and an abnormal range are set; identifying a plurality of determination candidate models each including a pattern for setting the normal range and the abnormal range for the examination item; calculating an accuracy corresponding to each of the plurality of determination candidate models based on model construction data corresponding to a disease related to the examination item; and determining, from the plurality of determination candidate models, a determination model that outputs whether determination data with respect to the examination item is normal or not based on the calculated accuracy.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 illustrates an exemplary health condition determination processing;

FIG. 2 illustrates exemplary examination data of a physical examination;

FIG. 3 illustrates an exemplary boundary value of examination data;

FIG. 4 illustrates an exemplary model construction processing;

FIG. 5 illustrates an exemplary range setting processing;

FIG. 6 illustrates an exemplary setting of a boundary range;

FIG. 7 illustrates an exemplary setting a boundary range;

FIG. 8 illustrates an exemplary health determination processing;

FIG. 9 illustrates an exemplary determination result;

FIG. 10 illustrates an exemplary determination result;

FIG. 11 illustrates an exemplary range setting processing;

FIG. 12 illustrates an exemplary setting a boundary range;

FIG. 13 illustrates an exemplary determination result;

FIG. 14 illustrates an exemplary range setting processing;

FIGS. 15A and 15B illustrate an exemplary setting a boundary range;

FIG. 16 illustrates an exemplary setting a boundary range;

FIG. 17 illustrates an exemplary health condition determination device;

FIG. 18 illustrates an exemplary computer;

FIG. 19 illustrates an exemplary model construction processing;

FIG. 20 illustrates an exemplary determination result;

FIG. 21 illustrates an exemplary determination result;

FIG. 22 illustrates an exemplary model construction processing;

FIG. 23 illustrates an exemplary normal range and an exemplary abnormal range; and

FIG. 24 illustrates an exemplary normal range and an exemplary abnormal range.

### DESCRIPTION OF EMBODIMENT

For example, future disease susceptibility is estimated based on examination data of a subject by considering hereditary data of the subject, attributes such as an age, or data of disease history. Posterior probability distribution of disease susceptibility of a subject is estimated by clustering distribution of a group and using distribution of disease susceptibility which is obtained through counting of every clustering node.

In a health instruction supporting system, examination items which are to be remedied are advised for every predetermined disease, based on a calculated relationship between a related examination item (checkup item) and disease onset, so as to avoid onset of the disease. An examination item which is related to disease contraction is found among a plurality of examination item data, based on statistical calculation based on values of respective data and actual disease contraction information.

For example, when contents of health instruction with respect to a subject are same as each other between a case where a value of examination data slightly exceeds a boundary value and a case where a value of examination data largely exceeds the boundary value, effectiveness of the health instruction may be degraded.

Whether a value of examination data which is related to a certain examination item of a physical examination of a subject is normal or abnormal is determined. Model construction processing and health determination processing using a constructed model are employed. The model construction processing or the health determination processing is performed. A device may be a general-purpose computer or a dedicated circuit. A part of a dedicated circuit may be combined with a general-purpose computer. A device includes at least a determination model construction unit which constructs a determination model for health determination and a determination unit which performs health determination of a subject based on examination data of a physical examination by using a determination model. The determination model construction unit executes model construction processing and the determination unit executes health determination processing.

The model construction processing includes boundary setting processing. Examination data for every examination item may be divided into three ranges which are a normal range, a boundary range, and an abnormal range. A normal range and an abnormal range are sectioned at a boundary value which is defined at international organizations or institutes based on medical knowledge. For every examination item of a physical examination, a range in which examination data is considered normal is set as a normal range and a range in which examination data is considered abnormal is set as an abnormal range. For example, when examination data related to an examination item is close to a boundary value even though the examination data is in an abnormal range, a disease related to the examination item may not be developed. Such range is set as a “boundary range” through model construction processing. A boundary range may include a boundary value which is defined based on medical knowledge and is present between a normal range in which examination data is considered normal and an abnormal range in which the examination data is considered abnormal. A boundary value may be positioned on a center of a boundary range and may not be positioned on the center. When a boundary range is set, a plurality of determination candidate models are created based on a determination of normal/abnormal with respect to data which belongs to the boundary range. A determination candidate model is selected from the plurality of determination candidate models through verification and the selected determination candidate model is set as a constructed determination model. A determination candidate model may be selected based on such criterion that which determination candidate model enables prediction of contraction of a disease with the highest accuracy, by using examination data of a plurality of past physical examinations to a subject and data of contraction of a disease of the subject (referred to as “learning data” or “model construction data” collectively). Two models may be obtained as a determination candidate model based on whether it is considered that a value, which belongs to one boundary range, for one examination item belongs to a normal range or to an abnormal range, for example. A determination candidate model may include a plurality of boundary ranges which are different from each other, for example. A determination candidate model may include a plurality of examination items. For example, a determination candidate model includes two examination items each of which has a single boundary range, and four models may be obtained based on whether it is considered that a value belonging to the boundary range belongs to a normal range or an abnormal range.

In the health determination processing, whether examination data (referred to as determination data, as well) of a subject related to an examination item of a physical examination is normal or abnormal is determined by using a determination model which is constructed through the model construction processing. For example, when there is low possibility that a subject will be affected by a disease related to an examination item, it may be considered that examination data of the subject is normal. For example, when there is high possibility that a subject will be affected by a disease related to an examination item, it may be determined that examination data is abnormal.

Thus, a determination model in which a specific property is considered with respect to an examination item is constructed. Through determination of a health condition with a determination model, a health condition of a subject related to whether possibility of onset of a specific disease is high or low, for example, may be determined with high accuracy.

In a health condition determination device which employs the above-mentioned health condition determination, a health condition of a subject of a physical examination may be determined with high accuracy. The health condition determination device may be realized by using a cloud server. Examination data for constructing a determination model may be widely collected and a health condition based on the examination data of a subject who has undergone a physical examination may be determined with high accuracy. According to the health condition determination device, advantages on aspects of cost and manpower may be obtained.

A method for setting a boundary range may include the following method.

(B1) A margin having a predetermined size, for example, a boundary range is set around a boundary value.

(B2) The boundary range is narrowed by considering distribution of model construction data such as learning data in the boundary range which is set in (B1).

(B3) The boundary range is set in consideration of disease contraction.

The size of a margin may be set in accordance with medical knowledge in (B1) or a margin may have a size in a predetermined rate of a boundary value. The predetermined rate of a boundary value may be 10%, 20%, or 30%, for example. A center of a boundary range may agree with a boundary value or a center of a boundary range may not agree with a boundary value. For example, when the size of a boundary range is 20% of the size of a boundary value, the boundary range may be expanded above and below by 10% respectively from the boundary value as the center. For example, when the size of a boundary range is 20% of the size of a boundary value, the boundary range may be expanded by 15% of the size of the boundary value toward a range having a larger value than the boundary value and may be expanded by 5% of the size of the boundary value toward a range having a smaller value than the boundary value.

In (B2), a boundary range may be narrowed by removing a range in which model construction data is not distributed in the boundary range from the boundary range, for example.

In (B3), a boundary value may be set by using a case in an examination history, for example, a case where a value of an examination result on a predetermined examination item is distributed around a boundary value and the value of the examination result is considered abnormal.

“A value of examination data is abnormal” may mean that it is possible to statistically or medically claim that examination data is related to contraction of a disease associated with an examination item. “A value of examination data is abnormal” may mean that it is possible to statistically or medically claim that examination data is related to disease susceptibility associated with an examination item.

In (B3), the minimum or maximum value which is not an outlier among examination data of a patient who is affected by a disease related to an examination item or the maximum or minimum value which is not an outlier among examination data of a patient who is not affected by a disease related to an examination item may be set as a boundary value.

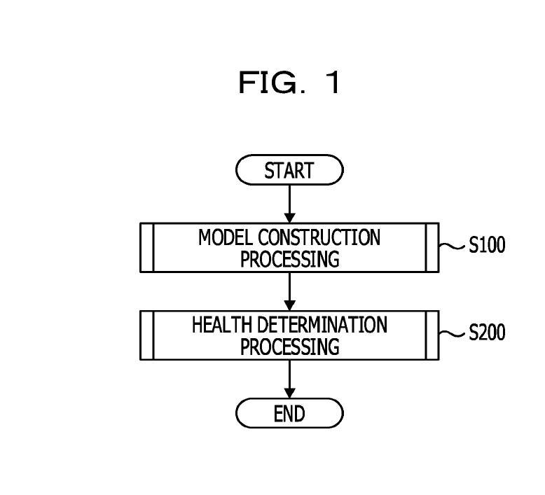

FIG. 1 illustrates an exemplary health condition determination processing.

In the health condition determination processing depicted in FIG. 1, model construction for health determination is performed in an operation S100.

“Health determination” may mean determining whether a subject is affected by a disease related to an examination item or whether disease susceptibility is high, based on a value of examination data of the examination item of the subject who has undergone a physical examination. Examples of a disease may include lifestyle-related diseases such as diabetes, metabolic syndrome, abnormal glucose tolerance, hypertension, and hyperlipidemia. Examples of an examination item may include an age, a body mass index (BMI), an abdominal girth, a blood-glucose level, gamma glutamyl transpeptidase (Γ-GTP), blood pressure, cholesterol, an insulin resistance index, plasma glucose, neutral fat, hepatic function (AST, IU/L), hepatic function (ALT, IU/L), adiponectin, glycoalbumin, free fatty acid, and insulin.

A “model” may have a function to output whether input examination data of an examination item of a health examination subject is “normal” or “abnormal”. A model may be referred to as a prediction model or a determination model. A model may be a mathematical model or a computing model for realizing algorithm to which supervised learning is applicable such as a neural network and a support vector machine, for example.

In “model construction”, the configuration or a parameter of a model may be set for a function of the model by using examination data of a plurality of persons. Further, a model may mean execution of supervised learning.

In the model construction of the operation S100, a boundary value which is defined at international organizations or institutes is verified based on actual examination data and the boundary value is changed as appropriate so as to increase correlation between examination data and disease contraction or high disease susceptibility. Examination data may be used as data for supervised learning of a model. For example, when a model which outputs whether input examination data is “normal” or “abnormal” includes a configuration or a parameter, the configuration or the parameter may be determined by using actual examination data so as to enhance the accuracy of output of the model.

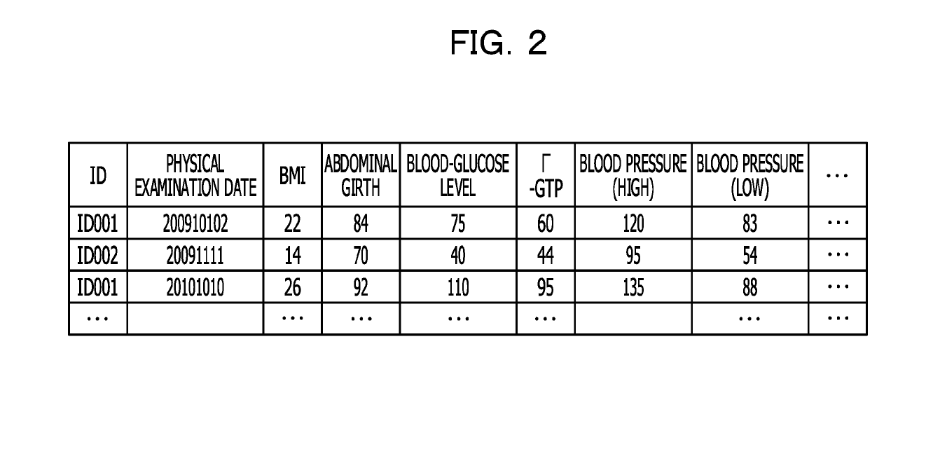

FIG. 2 illustrates exemplary examination data of a physical examination. The examination data depicted in FIG. 2 may be used in model construction.

In FIG. 2, a subject may be identified by an ID. Examination items may include a body mass index (BMI), an abdominal girth, a blood-glucose level, F-GTP, blood-pressure diastolic (or diastolic blood pressure, merely depicted as “blood pressure (low)” in FIG. 2), and blood-pressure systolic (systolic blood pressure, merely depicted as “blood pressure (high)” in FIG. 2).

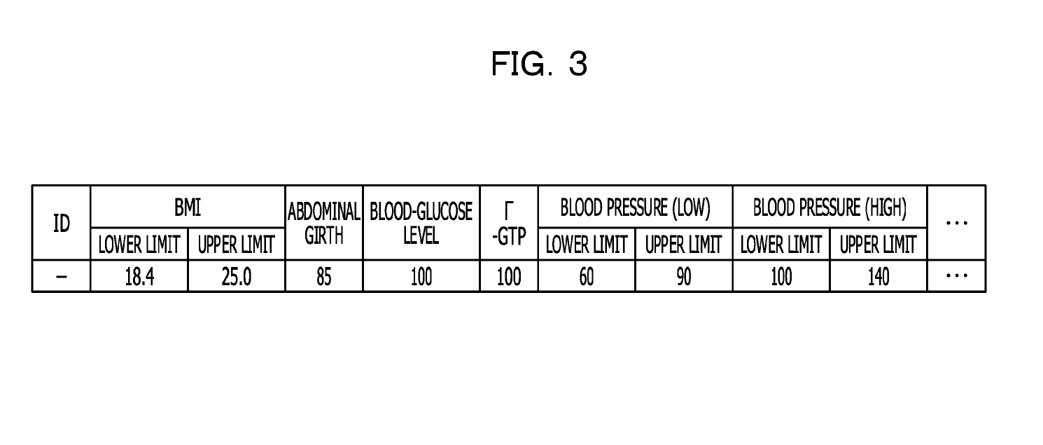

FIG. 3 illustrates exemplary boundary value of examination data. In FIG. 3, a boundary value of examination data, which is used for health determination, of a subject may be depicted. Examination items include an item with which examination data is determined normal or abnormal when the examination data belongs to a certain range and an item with which normal and abnormal are sectioned at a boundary value. The item with which examination data is determined normal or abnormal when the examination data belongs to a certain range may include a body mass index (BMI), blood-pressure diastolic, or blood-pressure systolic. The item with which normal and abnormal are sectioned at a boundary value may include an abdominal girth, a blood-glucose level, or F-GTP.

For example, regarding a body mass index (BMI), examination data is considered normal when being between 18.4 and 25.0, and examination data is considered abnormal when being smaller than 18.4 or larger than 25.0.

For example, regarding an abdominal girth, a value of examination data is considered normal when being smaller than 85 and a value of examination data is considered abnormal when being larger than 85.

In FIG. 1, when a model is constructed in the operation S100, health determination for determining whether a subject of a physical examination is healthy is performed by using the constructed model in an operation S200.

“Being healthy” may represent a state of being affected by no disease or a state with low disease susceptibility.

The health determination in an operation S200 may mean that whether input examination data of a certain examination item of a physical examination subject is “normal” or “abnormal” is output.

A determination model of which an output is “normal” or “abnormal” of input examination data of a physical examination of a subject is constructed. Health judgment is performed by using the constructed determination model. Therefore, health judgment may be simply performed.

The configuration, a parameter, or the like of a determination model is determined through supervised learning, so that a criterion which is different from criteria defined in institutes or the like may be set. For example, a special criterion may be set with respect to a group biased in sex ratio of patients of a physical examination, age groups, or life styles.

A determination model may be implemented on a calculator and determination may be automatically and effectively performed.

Boundary values depicted in FIG. 3 may be defined based on medical knowledge at international organizations or institutes, for example, so as to be widely referred. Data is determined by using an enormous number of samples so as to increase general versatility, so that updating may be delayed and the data may be unsuitable for a specific group.

When a relationship between examination data of an examination item and onset of a specific disease is considered, a boundary value of examination data of an examination item which is defined at international organizations or institutes, for example, may be unsuitable.

For example, even if examination data of an examination item slightly exceeds a boundary value to be judged abnormal in a physical examination of a certain year, the data may not be judged abnormal in the next year. For example, a gray zone existing around a boundary value may reduce the accuracy of health determination.

Model construction is performed by using actual examination data, so that a gray zone is reviewed with actual examination data and a boundary value is modified as appropriate. A determination model having a boundary value adapted to actuality may be obtained.

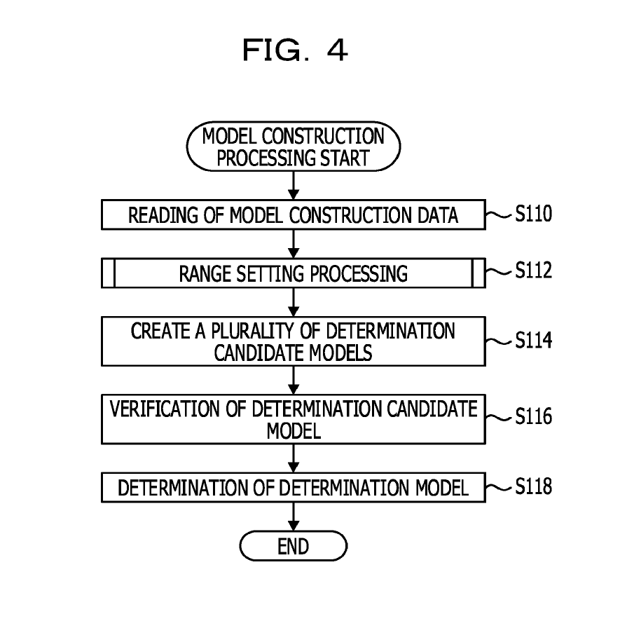

Model construction processing depicted in FIGS. 4 to 10 may correspond to the operation S100 depicted in FIG. 1, for example, and health determination processing may correspond to the operation S200 depicted in FIG. 1, for example. (B1) “a margin having a predetermined size, for example, a boundary range is set around a boundary value” may be employed for setting a boundary range, for example.

A predetermined size may be arbitrary, and may be 20% of a size of a boundary value, for example. A boundary range may be set in a manner to center a boundary value and a boundary range may not be symmetrically set around the boundary value. FIG. 4 illustrates an exemplary model construction processing.

In an operation S110, model construction data is read. For example, model construction data may be data depicted in FIG. 2. Model construction data is used as data for supervised learning from the viewpoint of construction of a model and may be referred to as learning data.

In an operation S112, range setting for a normal range, a boundary range, an abnormal range, and the like which are specified for every examination item is performed. A range which includes a boundary value and has a predetermined value width may be set as a boundary range, in a range of examination data with respect to an examination item, in which one or more boundary values for discriminating a normal range in which a value is considered normal and an abnormal range in which a value is considered abnormal are predetermined.

In an “abnormal range”, examination data in the range may be considered abnormal. In a “normal range”, a value of examination data may be considered not abnormal. A “boundary range” may be set close to a boundary value which is defined in international organizations, institutes, or the like. When examination data related to an examination item is in an abnormal range but close to a boundary value, for example, a “boundary range” may be a range in which a disease related to the examination item may not develop. When examination data related to an examination item is in a normal range but close to a boundary value, for example, a “boundary range” may be a range in which a disease related to the examination item may develop.

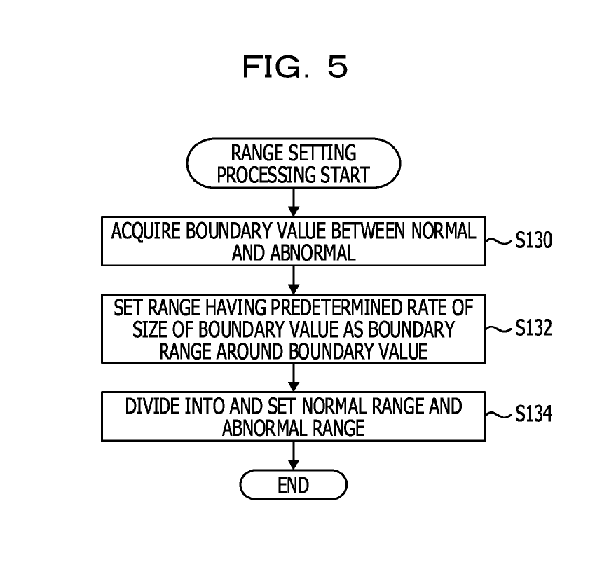

FIG. 5 illustrates an exemplary range setting processing. In an operation S130, a boundary value which is defined based on medical knowledge is obtained for every examination item of a physical examination. A boundary value sections a normal range and an abnormal range of a value of examination data of an examination item. A boundary value may be a value which is defined at international organizations, institutes, or the like.

In an operation S132, a range having a size in a predetermined rate of a size of a boundary value is set as a boundary range around the boundary value. A predetermined rate of a size of a boundary value may be 20%.

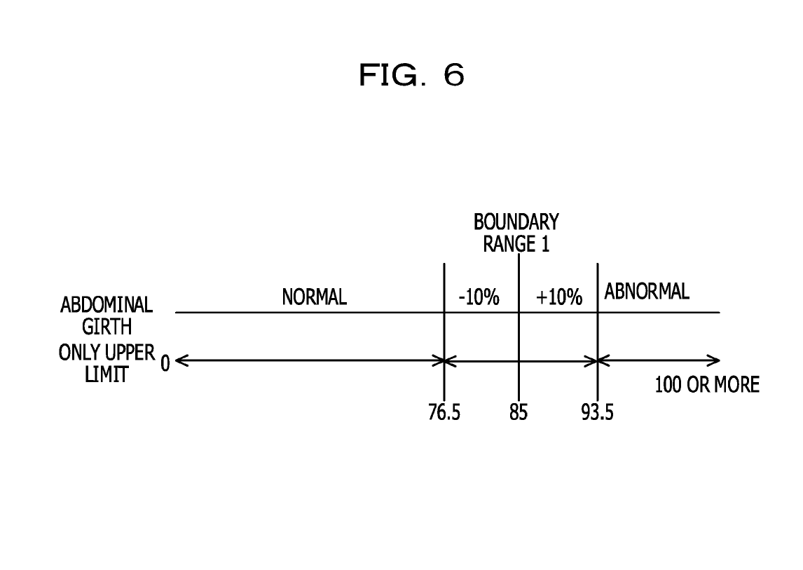

FIG. 6 illustrates an exemplary setting of a boundary range. In FIG. 6, a boundary range with respect to an abdominal girth may be set. For example, a boundary value of an abdominal girth may be 85 as depicted in FIG. 3. 20% of a size of a boundary value is 17. In a range which examination data related to an abdominal girth may take, a boundary range having a size of 17 may be set around 85 which is a boundary value. A set boundary range depicted in FIG. 6 covers values of examination data of an abdominal girth from 76.5 to 93.5 as a boundary range 1.

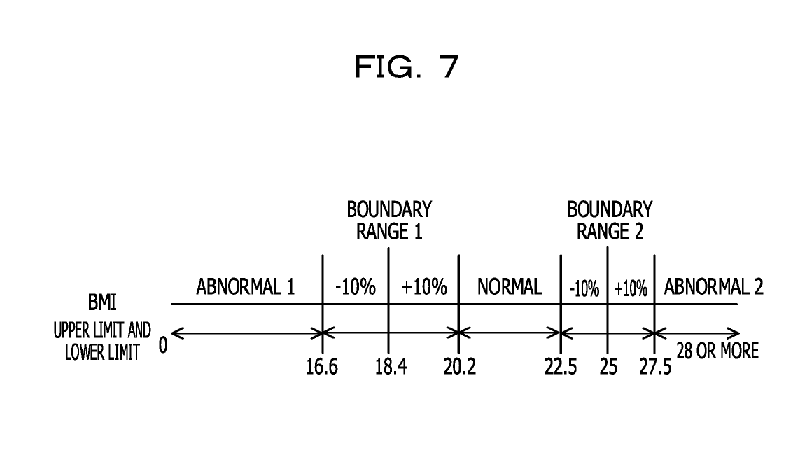

FIG. 7 illustrates an exemplary setting of a boundary range. In FIG. 7, a boundary range with respect to a body mass index (BMI) may be set. For example, as depicted in FIG. 3, a lower limit of a boundary value of a body mass index (BMI) may be 18.4 and an upper limit may be 25.0. Two boundary values are present, so that two boundary ranges may be set with respect to a body mass index (BMI). A boundary range 1 is for the lower limit boundary value which is 18.4 and may cover examination values from 16.6 to 20.2. A boundary range 2 is for the upper limit boundary value which is 25.0 and may cover examination values from 22.5 to 27.5.

In an operation S134 depicted in FIG. 5, a range other than a boundary range is divided into a normal range and an abnormal range to be set based on medical knowledge. For example, in FIG. 6, a range in which an abdominal girth is smaller than 76.5 is set as a normal range and a range in which an abdominal girth is larger than 93.5 is set as an abnormal range. Examination data belonging to these ranges may be respectively normal or abnormal irrespective of whether or not a boundary range is set. In FIG. 7, a range in which examination data of a body mass index (BMI) is smaller than 16.6 or a range in which examination data of a body mass index (BMI) is larger than 27.5 may be set as an abnormal range. A range between 20.2 and 22.5 may be set as a normal range.

The whole range which a value of examination data may take is divided into a normal range, a boundary range, and an abnormal range to be set.

In operation S114 depicted in FIG. 4, a plurality of determination candidate models which determine whether examination data belonging to a boundary range is considered normal or abnormal are created. For example, a plurality of patterns on setting of a normal range and an abnormal range with respect to an examination item may be created in accordance with whether a value included in the boundary range is considered normal or abnormal.

For example, in FIG. 6, examination data of an abdominal girth from 76.5 to 93.5 is covered as the boundary range 1. A model in which a value belonging to the boundary range 1 is considered normal and a model in which the value is considered abnormal are created. For example, three determination candidate models, in which regions in which a value of examination data is normal or abnormal are set, are created as following.

Model 1: normal=range on normal range side from boundary value, abnormal=range on abnormal range side from boundary value

Model 2: normal=normal range+boundary range 1, abnormal=abnormal range

Model 3: normal=normal range, abnormal=abnormal range+boundary range 1

In FIG. 7, three determination candidate models, in which regions in which examination data of a body mass index (BMI) is normal or abnormal are set, are created as following.

Model 1: normal=range on normal range side from boundary value, abnormal=range on abnormal range side from boundary value

Model 2: normal=normal range, abnormal=abnormal range 1+boundary range 1+boundary range 2+abnormal range 2

Model 3: normal=normal range+boundary range 2, abnormal=abnormal range 1+abnormal range 2+boundary range 1

Three determination candidate models may be created with respect to a body mass index (BMI). Five models which are a combination of four models in which examination data belonging to the boundary range 1 and the boundary range 2 are considered respectively in a normal range or an abnormal range and a model without a boundary range may be considered.

Models which output normal or abnormal with respect to respective input examination data with respect to a plurality of examination items may be considered. For example, models which output normal or abnormal with respect to respective input examination data of an abdominal girth and a body mass index (BMI) may be considered. Regarding models (determination models), 2³=8 patterns of determination candidate models in total may be considered. The 2³=8 patterns of determination candidate models are based on whether one boundary range with respect to examination data of an abdominal girth and two boundary ranges with respect to examination data of a body mass index (BMI) are respectively considered normal or abnormal.

In an operation S116, verification of a determination candidate model which is constructed in an operation S114 is performed. For example, the accuracy, which is calculated by using model construction data (learning data), with respect to respective determination candidate models may be compared to each other. The accuracy of determination of a determination candidate model may be verified based on predetermined information. The determination candidate model has a plurality of patterns related to setting of a normal range and an abnormal range and outputs whether model construction data is normal or abnormal based on an input of examination data with respect to an examination item. For example, predetermined information may be information related to whether or not a subject having examination data is affected by a specific disease related to an examination item or whether or not susceptibility to the specific disease is increased.

“Accuracy” may mean a magnitude of correlation between whether examination data with respect to individual examination items of a physical examination are normal or abnormal and whether a disease related to the examination items develops or does not develop.

Weighting may be performed with respect to respective determination candidate models so as to obtain the best accuracy.

In an operation S118, a determination model is determined. For example, when the accuracy is compared among respective determination candidate models, a determination candidate model having the highest accuracy is selected as a determination model. A determination model which outputs whether determination data is normal or abnormal may be selected from the plurality of determination candidate models, based on the accuracy of determination. The selected determination data is examination data that information related to whether or not a subject having examination data of an examination item is affected by a specific disease is not obtained.

For example, in a case where the accuracy of the model 1 is highest regarding an abdominal girth, examination data is determined normal when being smaller than 85 which is the boundary value and examination data is determined abnormal when being larger than 85.

In a case where the accuracy of the model 3 is highest regarding a body mass index (BMI), a value of examination data is determined abnormal when being smaller than 20.2 or larger than 27.5 and a value of examination data is determined normal when not being smaller than 20.2 or larger than 27.5.

Health determination of a subject of a physical examination is performed by using a determined determination model. For example, determination data such as examination data of an examination item is input into a determination model so as to determine whether the determination data is normal or abnormal.

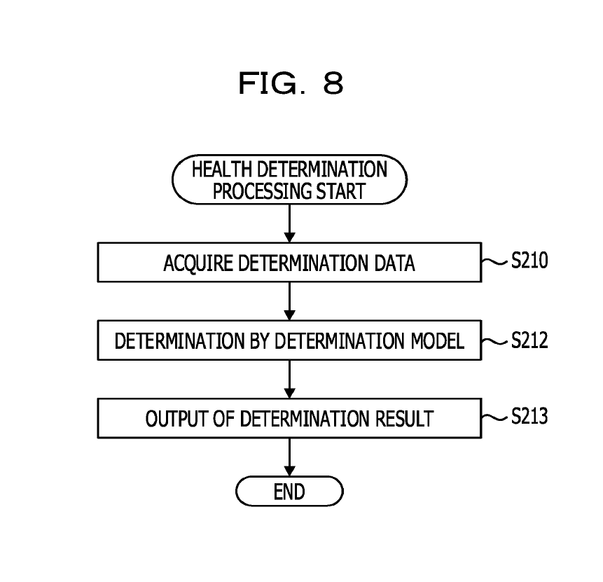

FIG. 8 illustrates an exemplary health determination processing. In an operation S210, determination data is acquired. “Determination data” may mean examination data of a physical examination with respect to a person who undergoes health determination. For example, examination data of an examination item may be obtained through a physical examination.

In an operation S212, the determination data which is acquired in operation S210 is input into a determination model so as to determine whether the determination data is normal or abnormal.

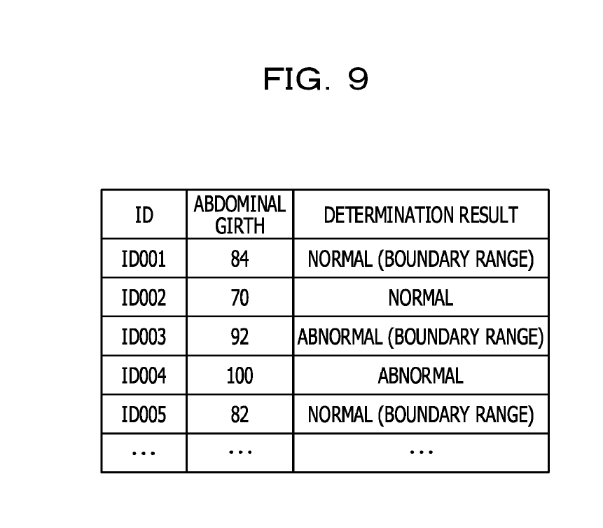

In an operation S214, a determination result is output. FIG. 9 illustrates an exemplary determination result. FIG. 9 illustrates a determination result with respect to an abdominal girth. IDs are imparted to respective subjects and the subjects are discriminated by IDs respectively. FIG. 9 illustrates determination results of whether values of examination data of abdominal girths of five persons having ID001 to ID005 respectively are normal or abnormal. For example, a value of examination data of an abdominal girth of a subject having ID001 is 84. A boundary value of an abdominal girth is 85 as depicted in FIG. 6. Although 84 which is a value of examination data of an abdominal girth of the subject having ID001 belongs to a boundary range close to a boundary value, it is determined normal since the value is smaller than 85 which is the boundary value. A determination result may be output so as to represent that examination data is in a boundary range.

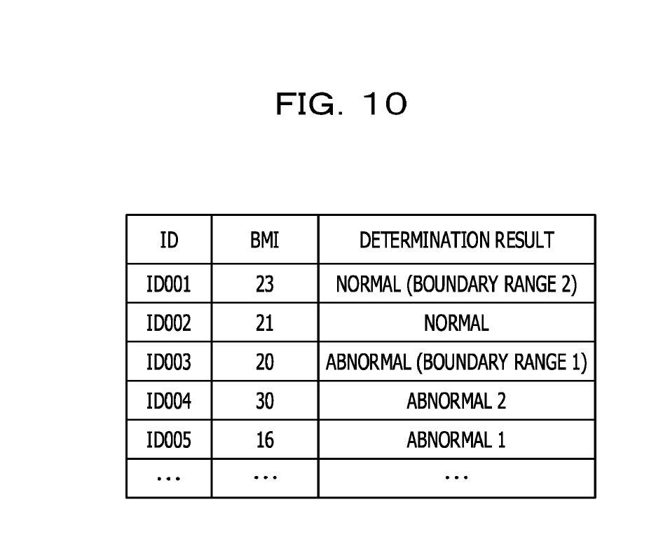

FIG. 10 illustrates an exemplary determination result. FIG. 10 illustrates a determination result with respect to a body mass index (BMI). Examination data is determined abnormal when being smaller than 20.2 or larger than 27.5 and a value of examination data is determined normal when not being smaller than 20.2 or larger than 27.5.

Model construction processing is performed based on actual examination data, so that a gray zone is examined by using actual examination data and a boundary value is modified as appropriate. A determination model having a boundary value adapted to actuality may be obtained. The width of a boundary range has a predetermined rate of a size of a boundary value, so that a boundary range may be easily set.

Examples of the model construction processing (operation S100 of FIG. 1) and the health determination processing (operation S200 of FIG. 1) are described. (B2) may be used as a method for setting a boundary range. For example, a boundary range may be narrowed in a manner to consider distribution of model construction data in a boundary range which is set as a margin having a predetermined size around a boundary value.

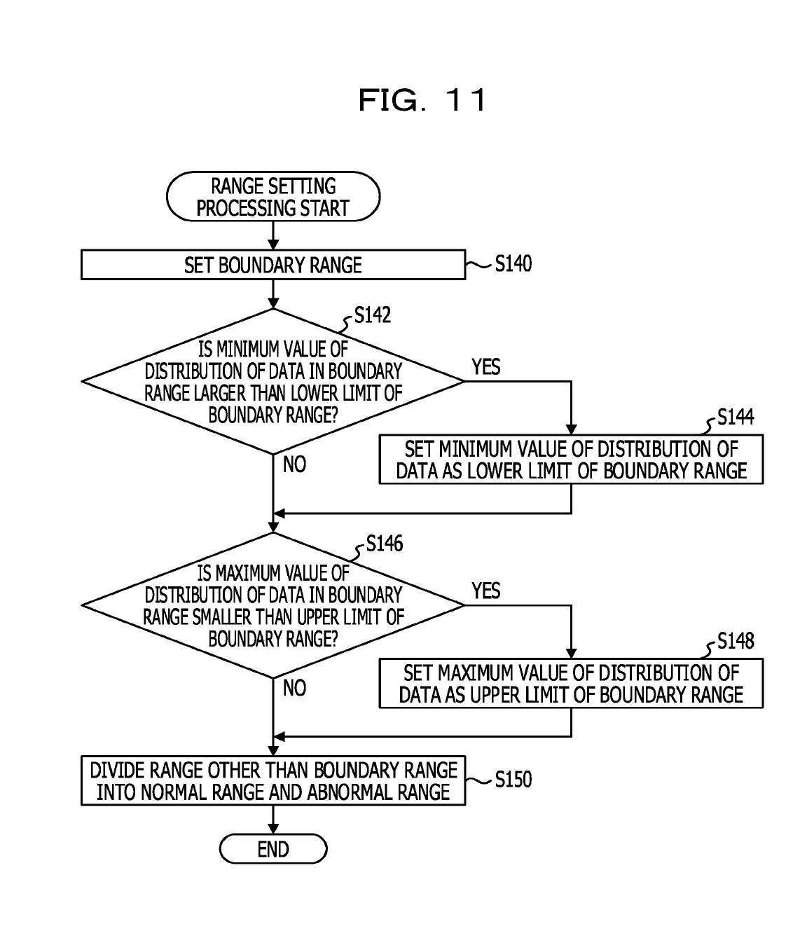

FIG. 11 illustrates an exemplary range setting processing. In an operation S140, a tentative boundary range is set. For example, a boundary range may be set as depicted in FIGS. 1 to 18.

When a boundary range is set, model construction data, for example, values of learning data may not be distributed in part of the boundary range. “Values are not distributed” may mean that a value of a distribution function is equal to or less than a predetermined value when arrangement of values of model construction data such as learning data are approximated by a continuous distribution function. “Values are not distributed” may mean that a range is not between the maximum value and the minimum value of model construction data in a boundary range.

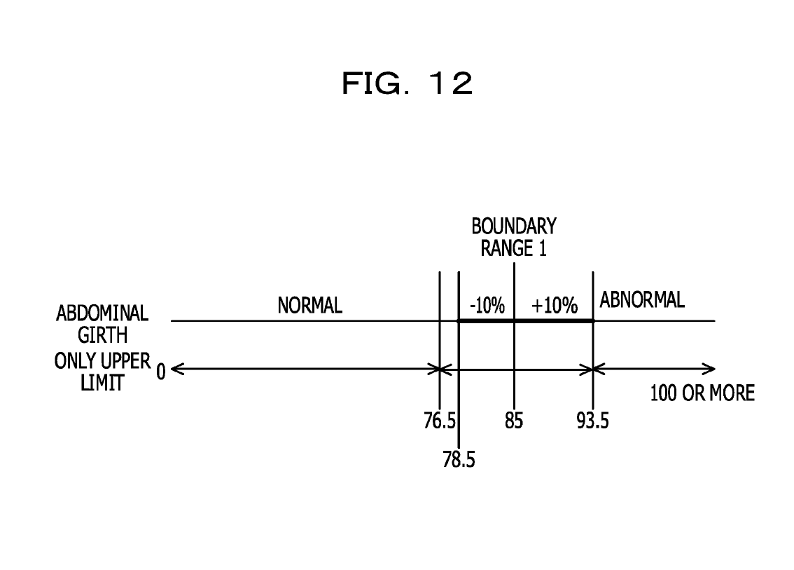

FIG. 12 illustrates an exemplary setting of a boundary range. When a boundary range with respect to an abdominal girth is set as depicted in FIGS. 1 to 18, distribution of model construction data may not exist between 76.5 and 78.5.

A boundary range with respect to an abdominal girth may be set as depicted in FIG. 12 and a boundary range may be set with respect to another examination item.

In an operation S142 depicted in FIG. 11, whether or not the minimum value of distribution of model construction data in the boundary range is larger than a lower limit of the boundary range is determined. When the determination result is Yes, for example, when the minimum value of the distribution of the model construction data in the boundary range is larger than the lower limit of the boundary range, the processing goes to an operation S144. When the determination result is No, for example, when the minimum value of the distribution of the model construction data in the boundary range is not larger than the lower limit of the boundary range, the processing goes to an operation S146.

In the operation S144, the minimum value of the distribution of the data is set as the lower limit of the boundary range. The processing goes to the operation S146.

The lower limit of the boundary range is 76.5 and the minimum value of the distribution of the model construction data in the boundary range is 78.5 in FIG. 12, so that the result of the determination in S142 is Yes. Through the processing of S144, 78.5 which is the minimum value of the data distribution is set as the lower limit of the boundary range.

In the operation S146, whether or not the maximum value of the distribution of the model construction data in the boundary range is smaller than an upper limit of the boundary range is determined. When the determination result is Yes, for example, when the maximum value of the distribution of the model construction data in the boundary range is smaller than the upper limit of the boundary range, the processing goes to an operation S148. When the determination result is No, for example, when the maximum value of the distribution of the model construction data in the boundary range is not smaller than the upper limit of the boundary range, the processing goes to an operation S150.

In the operation S148, the maximum value of the distribution of the data is set as the upper limit of the boundary range. The processing goes to the operation S150.

In the operation S150, a range other than the boundary range is divided into a normal range and an abnormal range based on medical knowledge, thus being set. Health determination of a subject of a physical examination may be performed by using a determined model.

The order of the operations S142 and S144 and the order of the operations S146 and S148 may be exchanged.

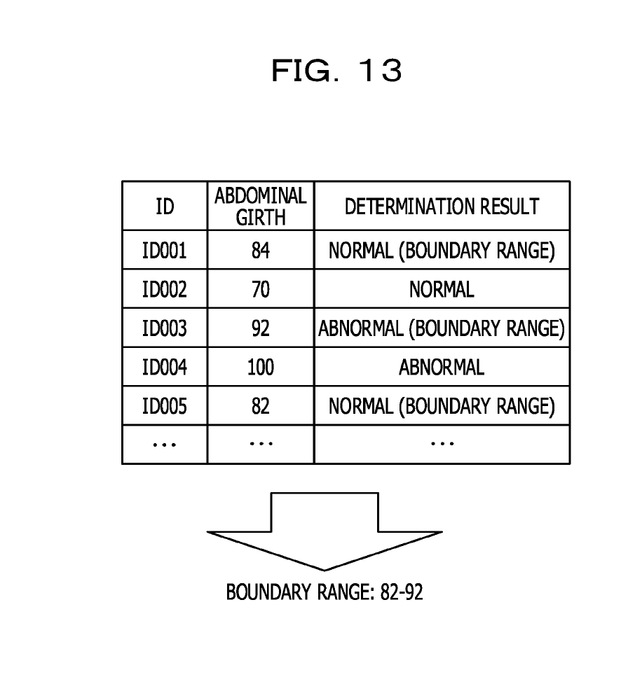

FIG. 13 illustrates an exemplary determination result. FIG. 13 illustrates model construction data of an abdominal girth, a boundary range which is determined based on the model construction data, and a determination result. In FIG. 13, the minimum value of the model construction data may be 82 which is examination data of a subject having ID005 and the maximum value may be 92 which is examination data of a subject having ID003, in the boundary range 1 which is set around a boundary value to have a range of 20% of a size of the boundary value, for example, set to be from 76.5 to 93.5 in FIG. 6. A range from 82 to 92 which are examination data of an abdominal girth is set as a boundary range. For example, the accuracy of the model 1 may be best among abdominal girths. For example, examination data of an abdominal girth may be determined normal when being smaller than 85 and examination data may be determined abnormal when being larger than 85. In a column of a determination result, normal and abnormal are sectioned at a boundary of 85 and information representing that examination data which is in a range from 82 to 92 is in the boundary range is added.

A specific property of physical examination data is reflected to setting of a boundary range, so that highly-accurate health determination may be performed.

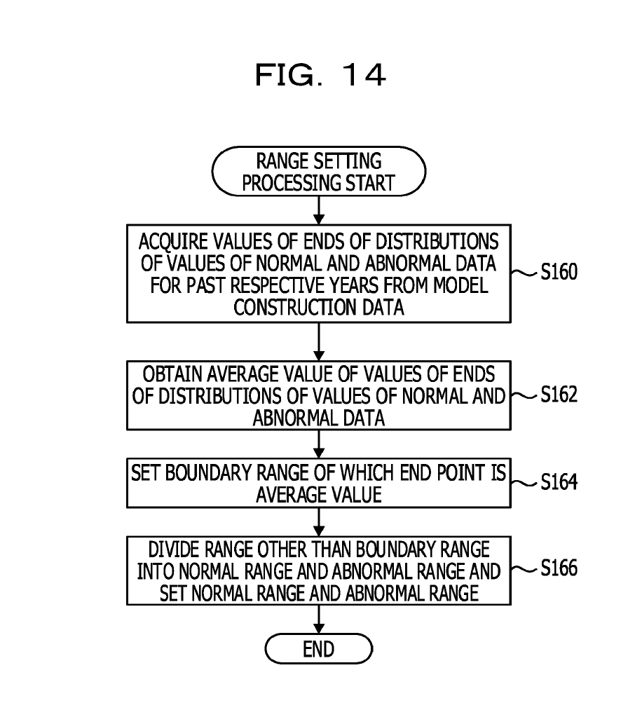

Model construction processing depicted in FIGS. 14 to 16 may correspond to the operation S100 depicted in FIG. 1, for example, and health determination processing may correspond to the operation S200 depicted in FIG. 1. The method of (B3) is applicable as a method for setting a boundary range. For example, a boundary range may be set by considering disease contraction. FIG. 14 illustrates an exemplary range setting processing.

When a value of examination data belongs to a boundary range, a case where examination data is abnormal and a case where examination data is normal are mixed and therefore, health determination is difficult. A model for determining a boundary range is constructed separately from a model for determining normal/abnormal, so that the accuracy may be improved. When a boundary range is adequately set in a range in which a case where examination data is abnormal and a case where examination data is normal are mixed, the accuracy may be improved.

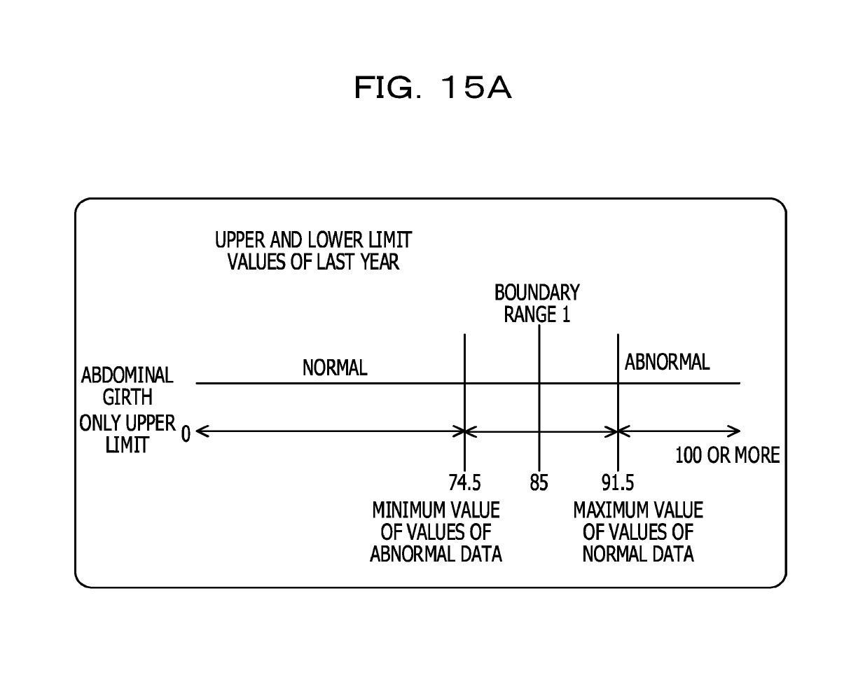

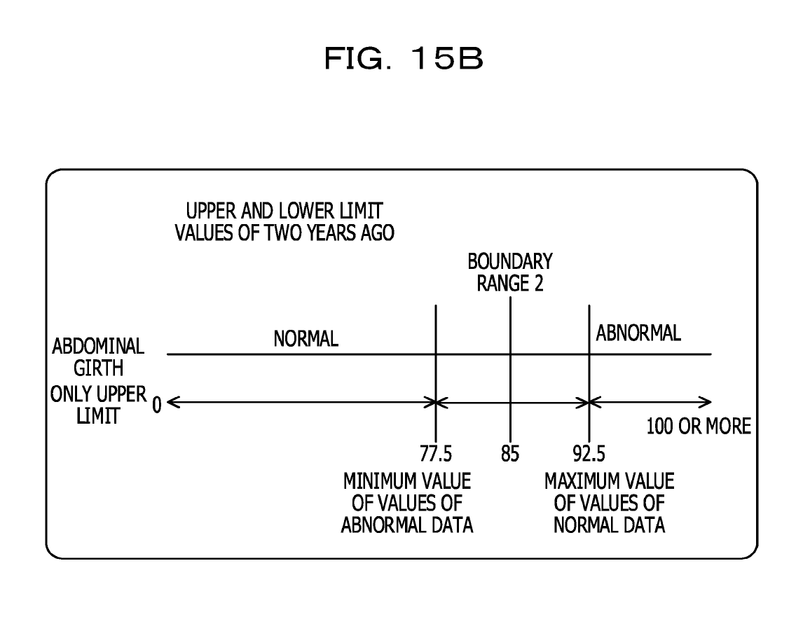

FIGS. 15A and 15B illustrate an exemplary setting of a boundary range. A boundary range is set by considering disease contraction. An examination item may be an abdominal girth.

FIG. 15A may illustrate a boundary range which is set based on examination data (model construction data) of the last year. The smallest value (the minimum value) among abnormal examination data, for example, the smallest value (the minimum value) among values of abdominal girths of subjects who have been affected by a disease or whose disease susceptibility has been increased from time of examination to present time may be 74.5. The largest value (the maximum value) among values of normal examination data, for example, the largest value (the maximum value) among abdominal girths of subjects who have not been affected by a disease or whose disease susceptibility has not been increased from time of examination to present time may be 91.5.

FIG. 15B may illustrate a boundary range which is set based on examination data (model construction data) two years ago. The smallest value (the minimum value) among values of abnormal examination data of abdominal girths may be 77.5 and the largest value (the maximum value) among normal examination data of abdominal girths may be 92.5.

In every year, the smallest value (the minimum value) among abnormal values of abdominal girths of examination data (model construction data) and the largest value (the maximum value) among normal values of abdominal girths of examination data have variation. A value of an end of distribution of abnormal examination data in examination data such as model construction data and a value of an end of distribution of normal examination data may be obtained in every year so as to set an average of values of ends of distribution of years as a boundary range.

FIGS. 15A and 15B illustrate the smallest value which is not an outlier among values of abnormal examination data based on examination data of abdominal girths of last year or two years ago and the largest value which is not an outlier among values of normal examination data. For example, examination data n years ago (n is an integer larger than 2) may be used.

In FIGS. 15A and 15B, a value smaller than a boundary value is determined normal and a value larger than the boundary value is determined abnormal. Regarding an examination item in which a value larger than a boundary value is determined normal and a value smaller than the boundary value is determined abnormal, the largest value among values of abnormal examination data and the smallest value among values of normal examination data are used.

In the operation S160 depicted in FIG. 14, a value of an end of distribution of values of abnormal and normal examination data of every past year is obtained based on past examination data such as model construction data. A “value of an end of distribution” may be a value which is not an outlier among abnormal and normal examination data related to an examination item. For example, an outlier is obtained by outlier=average value±(3×standard deviation), with respect to statistics to which an average value and a standard deviation are imparted.

When an examination item is an abdominal girth, the smallest value (the minimum value) which is not an outlier among abnormal values of an abdominal girth and the largest value (the maximum value) which is not an outlier among normal values of an abdominal girth may be obtained.

In an operation S162, an average value of a point of an end of distribution of normal or abnormal examination data is obtained.

For example, the minimum value of values of abnormal examination data of last year may be Min(1) and the minimum value of values of abnormal examination data two years ago may be Min(2), and the minimum value of values of abnormal examination data n years ago may be Min(n). At this time, an average of the minimum values may be obtained by the following formula.

$$Min = \frac{{\sum }_{i = 1}^{n} Min ( i )}{n}$$

The maximum value of normal examination data of last year may be Max(1), the maximum value of values of normal examination data two years ago may be Max(2), and the maximum value of values of normal examination data n years ago may be Max(n). At this time, an average of the maximum values may be obtained by the following formula.

$$Max = \frac{{\sum }_{i = 1}^{n} Max ( i )}{n}$$

In FIGS. 15A and 15B, the minimum value is obtained as following.

Min=(minimum value among values of abnormal examination data of last year+minimum value among values of abnormal examination data two years ago)/2

=(74.5+77.5)/2=76

Max=(maximum value among values of normal examination data of last year+maximum value among values of normal examination data two years ago)/2

=(91.5+92.5)/2=92

In an operation S164, a boundary range of which a lower limit and an upper limit are respectively Min and Max which are obtained in the operation S162 is set.

In an operation S166, a range other than the boundary range is divided into a normal range and an abnormal range based on medical knowledge, thus being set.

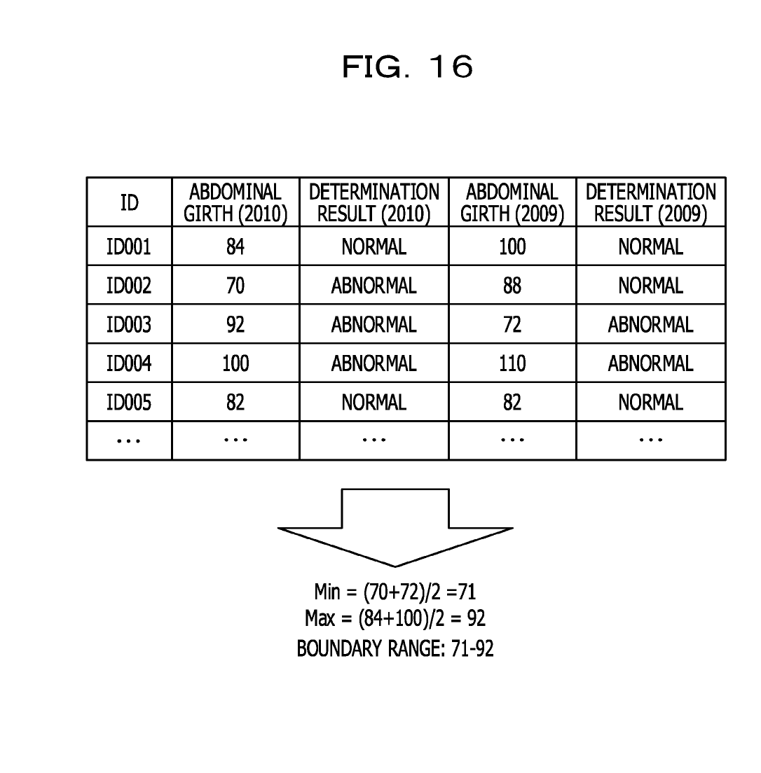

FIG. 16 illustrates an exemplary setting of a boundary range. In FIG. 16, a boundary range of an abdominal girth may be set. In examination data of 2010, the minimum value among values of abnormal examination data may be 70 and the maximum value among values of normal examination data may be 84. In examination data of an abdominal girth of 2009, the minimum value among values of abnormal examination data may be 72 and the maximum value among values of normal examination data may be 100. An average value of the minimum values of examination data of years and an average value of the maximum values are respectively Min=(70+72)/2=71 and Max=(84+100)/2=92. In FIG. 16, a lower limit of the boundary range is set to 71 and an upper limit is set to 92.

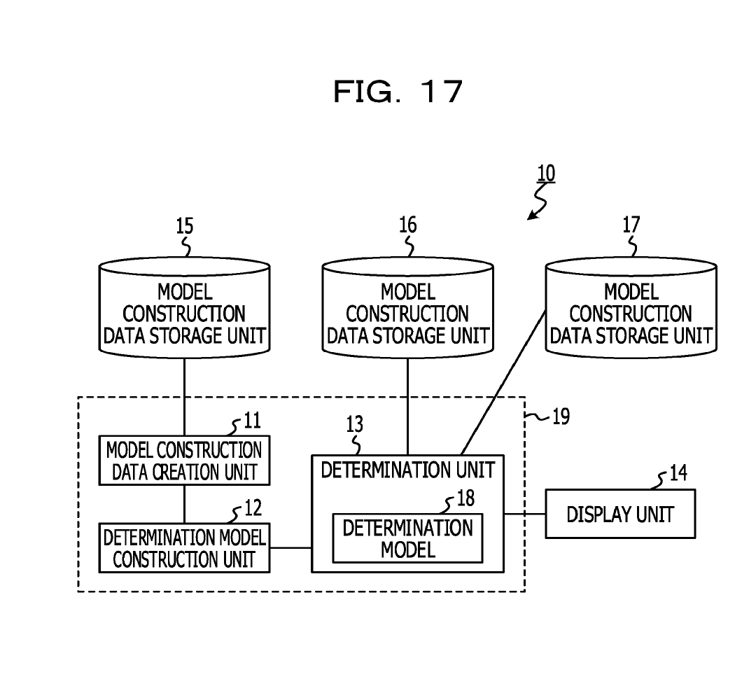

FIG. 17 illustrates an exemplary health condition determination device. A health condition determination device 10 may refer to model construction data which is stored in a model construction data storage unit 15, determination result data which is stored in a determination result data storage unit 17, and determination data which is stored in a determination data storage unit 16.

Model construction data may be examination data of an examination item of a subject of a physical examination, for example.

A model construction data creation unit 11 creates model construction data by using examination data of a past physical examination, and the like. For example, examination data of an examination item of a subject of a physical examination is organized for each identifier (ID) of a subject of the physical examination. Examination data of a subject for every examination item may be prepared for model construction data.

Examination data, which has specific attribution, of a subject may be extracted from examination data of the subject of a physical examination so as to be set as model construction data. “Attribution” includes age, sex, occupation, residence, food preference, life pattern, and the like. Food preference may indicate whether to like sweets, for example. Life pattern may indicate whether or not to smoke, length of sleeping time, wake-up time, time of sleep, time and way of commuting, hobby, and the like. Hobby may indicate whether or not to sport, for example.

The determination model construction unit 12 constructs a determination model 18 by using model construction data which is created in the model construction data creation unit 11. “To construct a determination model” may mean performing “model construction”. In “model construction”, the configuration or a parameter of a model for realizing a function which is to be owned by the model may be set by using examination data of a plurality of persons. When a model is a mathematical model or a computing model for realizing algorithm to which supervised learning is applicable, “model construction” may mean supervised learning.

For example, in the determination model construction unit 12, the model construction processing depicted in FIG. 14 may be performed.

A determination unit 13 determines whether a value of examination data, which is input from the determination data storage unit 16, of a subject on an examination item is normal or abnormal, by using the determination model 18 which is constructed in the determination model construction unit 12.

For example, in the determination unit 13, the health determination processing depicted in FIG. 8 may be performed. A result of determination in the determination unit 13 is transmitted to the determination result data storage unit 17 and a display unit 14. A result of determination may be displayed to a subject.

All or part of the model construction data creation unit 11, the determination model construction unit 12, and the determination unit 13 may be provided as a cloud server 19. When all of the model construction data creation unit 11, the determination model construction unit 12, and the determination unit 13 are provided as the cloud server 19, the model construction data storage unit 15 and the determination data storage unit 16 may be included in a terminal which is coupled to the cloud server 19. The determination result data storage unit 17 and/or the display unit 14 may be included in the terminal as well. When the model construction data creation unit 11 and the determination model construction unit 12 are provided as the cloud server 19, the determination data storage unit 16 is included in the terminal which is coupled to the cloud server 19.

Only the determination unit 13 may be included in the cloud server 19 and the model construction data creation unit 11 and the determination model construction unit 12 may be included in a terminal which is coupled to the cloud server 19.

The determination model construction unit 12 depicted in FIG. 17 includes a boundary range setting unit, a range setting unit, a model verification unit, and a model determination unit.

The boundary range setting unit may set a range which includes a boundary value and has a predetermined width as a boundary range in a range of examination data with respect to an examination item that includes one or more boundary values which section a normal range in which a value is considered normal and an abnormal range in which a value is considered abnormal.

The range setting unit may create a plurality of patterns on setting of a normal range and an abnormal range with respect to an examination item, based on whether a value included in a boundary range is considered normal or abnormal.

When examination data of an examination item of a plurality of determination candidate models having a plurality of patterns on setting of a normal range and an abnormal range is input, the model verification unit calculates the accuracy of determination of a determination candidate model for outputting whether a value of model construction data is normal or abnormal, based on information indicating whether or not a subject having the examination data is affected by a disease related to the examination item, so as to verify the determination candidate model.

When determination data which is examination data of an examination item and is examination data in which information on whether or not a subject having the examination data is affected by a disease is not obtained is input, the model determination unit determines a determination model for outputting whether the value of the determination data is normal or abnormal from a plurality of determination candidate models based on the accuracy of determination. The determination unit 13 depicted in FIG. 17 inputs determination data which is examination data of an examination item to a determination model and determines whether the determination data is normal or abnormal. The determination unit 13 includes an output unit which outputs a determination result to the determination result data storage unit 17 and/or the display unit 14.

The determination result data storage unit 17 or the display unit 14 that are depicted in FIG. 17 outputs a determination result which is output from the determination unit 13 to a user.

A function block of the device depicted in FIG. 17 may be configured by a computer having the hardware configuration.

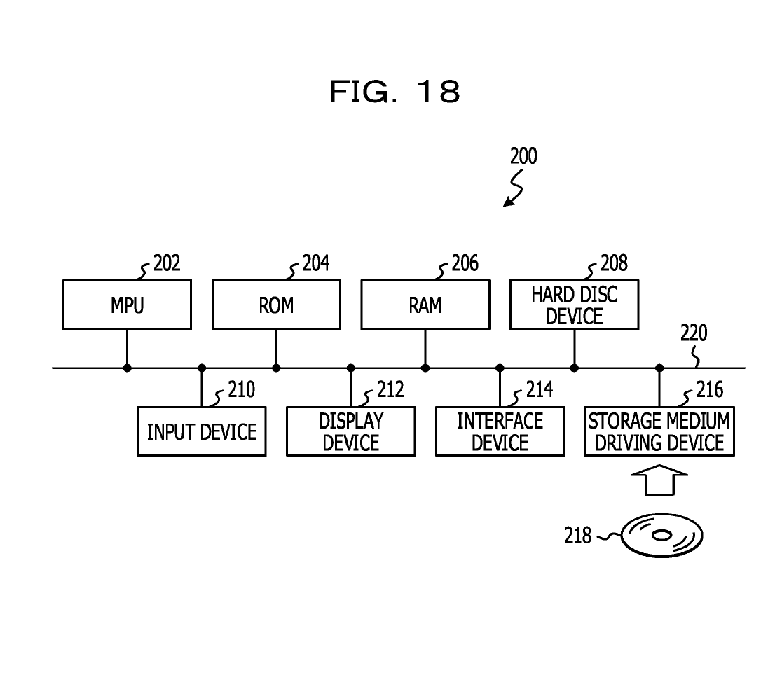

FIG. 18 illustrates an exemplary computer. The computer depicted in FIG. 18 may be used by the health condition determination device 10 depicted in FIG. 17.

A computer 200 includes a micro processing unit (MPU) 202, a read only memory (ROM) 204, a random access memory (RAM) 206, a hard disc device 208, an input device 210, a display device 212, an interface device 214, and a storage medium driving device 216. These elements may be coupled with each other via a bus line 220 and data may be mutually transmitted/received under the control of the MPU 202.

The MPU 202 may be an arithmetic processing device which controls the computer 200 and may be a control processing unit of the computer 200.

The ROM 204 may be a readout dedicated semiconductor memory which stores a predetermined basic control program. The MPU 202 reads out and executes the basic control program at start-up of the computer 200 so as to control an operation of elements of the computer 200.

The RAM 206 may be a semiconductor memory which is writable and readable as demanded and is used as an operation storage region as appropriate when the MPU 202 executes the control program.

The hard disc device 208 may be a storage device which stores a control program or data which is executed by the MPU 202. The MPU 202 may reads out and executes the control program which is stored in the hard disc device 208 so as to execute control processing.

The input device 210 may be a mouse device or a keyboard device, for example. When the input device 210 is operated by a user of the system depicted in FIG. 6, the input device 210 acquires information associated with an operation content and transmits acquired input information to the MPU 202.

The display device 212 may be a liquid crystal display, for example, and display a text or an image in response to display data which is transmitted from the MPU 202.

The interface device 214 performs administration of transmission/reception of information with respect to equipment which is coupled to the computer 200.

The storage medium driving device 216 may be a device which reads out a control program and data which are stored in a transportable storage medium 218. The MPU 202 may read out and execute the control program which is stored in the transportable storage medium 218 via the storage medium driving device 216, whereby executing control processing. The transportable storage medium 218 may include a flash memory including a connector of the universal serial bus (USB) standard, a compact disc read only memory (CD-ROM), a digital versatile disc read only memory (DVD-ROM), and the like, for example.

When the health condition determination device 10 depicted in FIG. 17 is configured by using the computer 200, a control program for making the MPU 202 perform processing of the model construction data creation unit 11, the determination model construction unit 12, or the determination unit 13, for example, is created. The created control program may be preliminarily stored in the hard disc device 208 or the transportable storage medium 218. The MPU 202 reads out and executes the control program based on a predetermined instruction. A function included in the health condition determination device 10 depicted in FIG. 17 is provided by the MPU 202. The computer 200 functions as the health condition determination device 10 depicted in FIG. 17.

In setting of a boundary range, (B4) a method of setting a plurality of boundary ranges in a stepwise fashion may be included. A boundary range is changed in a stepwise fashion, so that the accuracy may be improved. A range which includes a boundary value and has a width of a predetermined rate of a size of the boundary value, for example, 20% of the size of the boundary value is set as a boundary range. A determination candidate model is constructed using a boundary range which is set. The prediction accuracy of a determination candidate model is obtained. By narrowing a boundary range, another determination candidate model is constructed and the accuracy thereof is obtained. For example, the width of a boundary range is set to be 18% of the size of a boundary value. A range of a boundary range may be gradually narrowed, the accuracy of a determination candidate model having each boundary range may be obtained, and a determination candidate model having a boundary range of the best accuracy may be employed as a determination model.

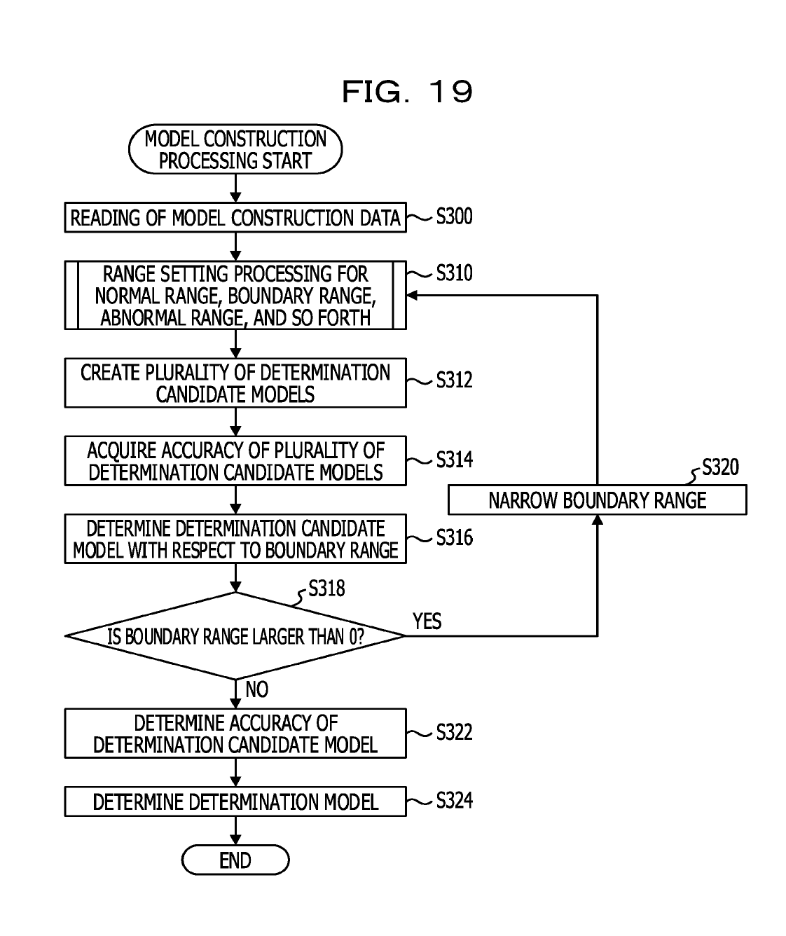

FIG. 19 illustrates an exemplary model construction processing. In an operation S300, model construction data (learning data) is read in. The processing of the operation S300 may be substantively same as or similar to the operation S110 depicted in FIG. 4.

In an operation S310, range setting processing for setting a normal range, a boundary range, or an abnormal range for each examination item of a physical examination is executed.

When the operation S310 is executed for the first time, a range which includes a boundary value and has 20% of a size of the boundary value may be set as a boundary range, for example. The method (B1) may be used, for example. The methods (B2) and (B3) may be used. In the operation S310, a range other than the boundary range is divided into a normal range and an abnormal range based on medical knowledge.

An initial value of the width of the boundary range may be arbitrarily set by an operator. An initial value may be 20% of a boundary value, may be wider such as 40%, or may be narrower such as 10%.

When the operation S310 is executed on the second time and after, a boundary range corresponding to the width of the boundary range which is defined in an operation S320 is set, and a range other than the boundary range is divided into a normal range and an abnormal range based on medical knowledge.

In an operation S312, a plurality of determination candidate models are created based on whether examination data belonging to the boundary range is considered normal or abnormal. Processing of the operation S312 may be substantively same as or similar to the processing of an operation S114 depicted in FIG. 4. The processing goes to an operation S314.

In the operation S314, the accuracy of each of the plurality of determination candidate models which are created in the operation S312 is acquired.

In an operation S316, a determination candidate model which provides the highest accuracy among accuracies acquired in the operation S314 is selected as a determination candidate model with respect to a boundary range which is set. The processing goes to an operation S318.

In the operation S318, whether or not the boundary range is larger than 0 is determined. When the determination is Yes, for example, when the boundary range is larger than 0, the processing goes to an operation S320. When the determination is No, for example, when the boundary range is 0 or less, the processing goes to an operation S322.

In the operation S320, a boundary range is narrowed. An operator may arbitrarily set a narrowing unit, for example, may set a value of 1%, 2%, or 0.5%. The processing goes to operation S310.

In the operation S322, a determination candidate model having the highest accuracy among a plurality of determination candidate models which are constructed with respect to widths of a plurality of boundary ranges is determined. Among a plurality of determination candidate models having a plurality of patterns related to setting of a normal range and an abnormal range, when examination data of an examination item is input, the accuracy of determination of a determination candidate model for outputting whether model construction data is normal or abnormal may be calculated based on information related to whether or not a subject having the examination data is affected by a disease related to the examination item or whether or not disease susceptibility is increased, and thus a determination candidate model may be verified.

In an operation S324, a determination candidate model having the highest accuracy is selected as a determination model. A determination model for outputting whether determination data is normal or abnormal may be determined, from a plurality of determination candidate models, based on the accuracy of determination which is calculated in the operation S322. The determination data is examination data of an examination item and is examination data in which information on whether or not a subject having the examination data is affected by a specified disease or whether or not disease susceptibility is increased is not obtained. Weighting may be performed with respect to each model so as to obtain the best accuracy.

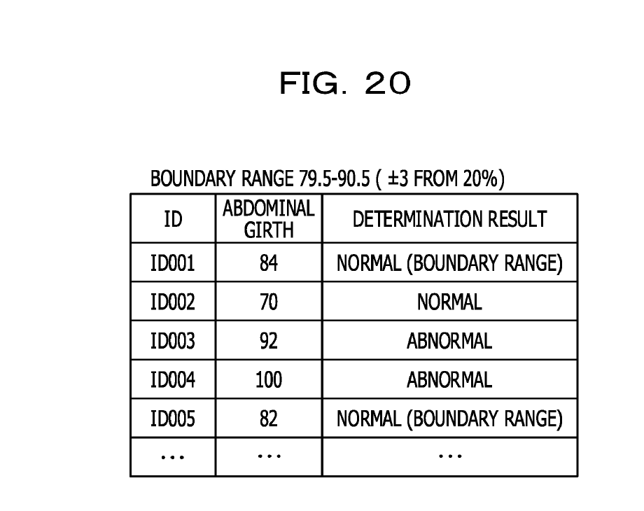

FIG. 20 illustrates an exemplary determination result. FIG. 20 illustrates a determination result which is obtained when a boundary range is narrowed by decreasing an upper limit by −3 and increasing a lower limit by +3, from the upper limit and the lower limit of an initial boundary range which is set as a region having 20% of a size of a boundary value.

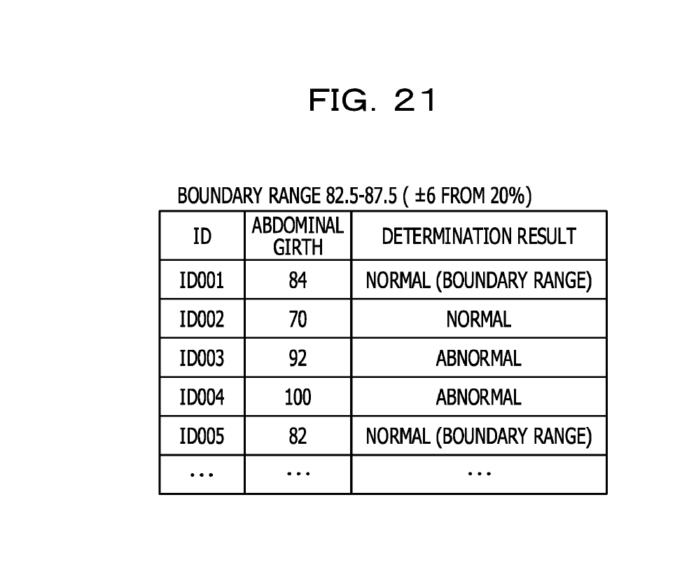

FIG. 21 illustrates an exemplary determination result. A determination result which is obtained when a boundary range is narrowed by decreasing an upper limit by −6 and increasing a lower limit by +6, from the upper limit and the lower limit of an initial boundary range which is set as a region having 20% of a size of a boundary value is illustrated. The determination result depicted in FIG. 20 is different from the determination result depicted in FIG. 21. A determination result changes depending on a range in which a boundary range is set.

Widths of a plurality of values which are obtained by decreasing a predetermined initial value in a stepwise fashion are set as the size of the boundary range, thus determining a model. Therefore, highly-accurate health condition determination may be realized.

The above-described setting of a boundary range may be executed by the device depicted in FIG. 17 or 18.

A range in which a boundary range is not set and which may be taken by examination data for every examination item may be divided into two ranges which include a normal range and an abnormal range based on a boundary value based on medical knowledge.

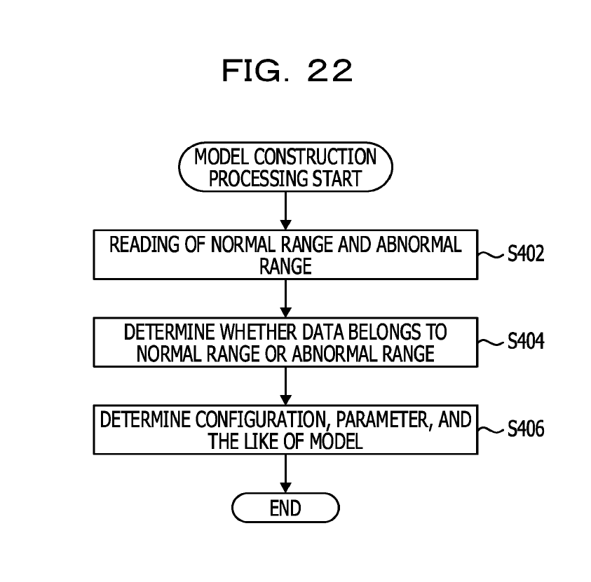

FIG. 22 illustrates an exemplary model construction processing. In an operation S402, a normal range and an abnormal range which are defined based on medical knowledge for every item are read in.

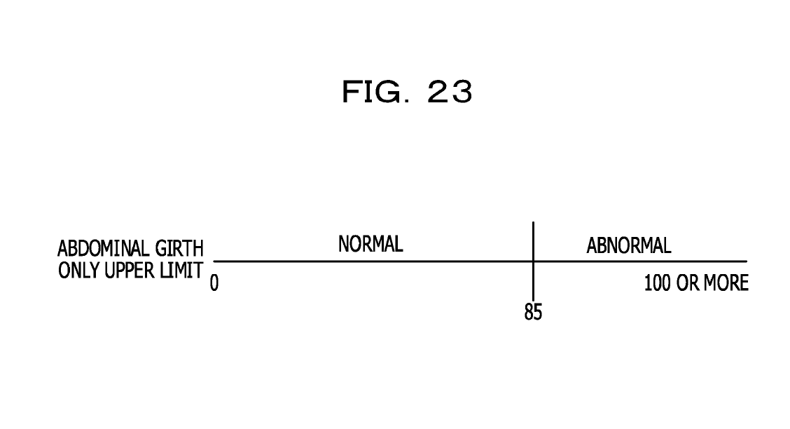

FIG. 23 illustrates an exemplary normal range and an exemplary abnormal range. FIG. 23 may illustrate a normal range and an abnormal range with respect to an abdominal girth. For example, a range which has a value smaller than 85 and in which “normal” is depicted may be a normal range. A range which has a value larger than 85 and in which “abnormal” is depicted may be an abnormal range.

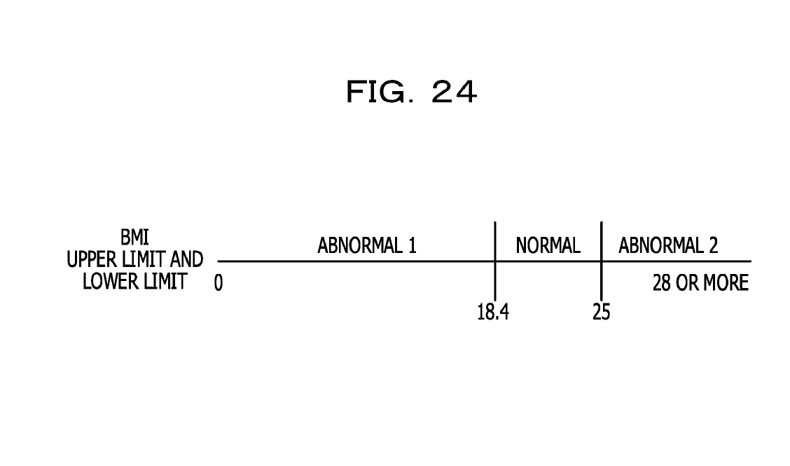

FIG. 24 illustrates an exemplary normal range and an exemplary abnormal range. FIG. 24 may illustrate a normal range and an abnormal range with respect to a body mass index (BMI). A range which has a value smaller than 18.4 and in which “abnormal 1” is depicted and a range which has a value larger than 25 and in which “abnormal 2” is depicted may be abnormal ranges. A range which is between 18.4 and 25 and in which “normal” is depicted may be a normal range.

In an operation S404, whether individual data belongs to a normal range or an abnormal range is determined based on an item and data.

In an operation S406, the configuration, a parameter, and the like of a model is determined and a model is determined.

A case where examination data slightly exceeds a boundary value and a case where examination data largely exceeds the boundary value may have substantially the same determination of whether the examination data is normal or abnormal. Therefore, the accuracy of determination or effectiveness of health instruction may be degraded.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. An information processing apparatus comprising:
a memory configured to store predetermined value independently for each of a plurality of determination items, the predetermined value indicating boundary value to distinguish normal value and abnormal value; and
a processor coupled to the memory and configured to
store, for each of the determination items, range information indicating first range and second range in the memory in association with corresponding determination item, the first range being independently set for each of the plurality of determination items and variably set for a range between the predetermined value and a first value which is identified as the normal value in comparison with the predetermined value, the second range being independently set for each of the plurality of determination items and variably set for a range between the predetermined value and a second value which is identified as the abnormal value in comparison with the predetermined value,
store, for each of the determination items, model information that indicates which one of a first model, a second model, and a third model is to be applied to the corresponding determination item in the memory in association with corresponding determination item, the first model being used to determine a value included in the first range as the normal value and to determine a value included in the second range as the abnormal value, the second model being used to determine a value included in the first range as the normal value and to determine a value included in the second range as the normal value, the third model being used to determine a value included in the first range as the abnormal value and to determine a value included in the second range as the abnormal value, such that information used to determine the normal value and the abnormal value is able to be optimized for each of the plurality of determination items by separately storing the range information and the model information in the memory,
obtain, in response to receiving data corresponding to one of the plurality of determination items, the range information and the model information separately from the memory, each of the range information and the model information corresponding to the determination item of the received data,
determine, based on the separately obtained range information and the model information, whether a value included in the received data is normal value or abnormal value, and
output result of the determination.

2. The information processing apparatus according to claim 1, wherein
the processor is configured to determine, when the value of the received data is included in one of the first range and the second range, whether the value included in the received data is normal value or abnormal value based on corresponding determination model among the first, second, and third model indicated by the model information.

3. The information processing apparatus according to claim 1, wherein
the processor is configured to determine, when the value of the received data is not included in any of the first range or the second range, whether the value included in the received data is normal value or abnormal value by comparing the value with one of the first value and the second value.

4. A method comprising:
storing, by a processor, in a memory, predetermined value independently for each of a plurality of determination items, the predetermined value indicating boundary value to distinguish normal value and abnormal value; and
storing, by the processor, for each of the determination items, range information indicating first range and second range in the memory in association with corresponding determination item, the first range being independently set for each of the plurality of determination items and variably set for a range between the predetermined value and a first value which is identified as the normal value in comparison with the predetermined value, the second range being independently set for each of the plurality of determination items and variably set for a range between the predetermined value and a second value which is identified as the abnormal value in comparison with the predetermined value,
storing, by the processor, for each of the determination items, model information that indicates which one of a first model, a second model, and a third model is to be applied to the corresponding determination item in the memory in association with corresponding determination item, the first model being used to determine a value included in the first range as the normal value and to determine a value included in the second range as the abnormal value, the second model being used to determine a value included in the first range as the normal value and to determine a value included in the second range as the normal value, the third model being used to determine a value included in the first range as the abnormal value and to determine a value included in the second range as the abnormal value, such that information used to determine the normal value and the abnormal value is able to be optimized for each of the plurality of determination items by separately storing the range information and the model information in the memory,
obtaining, by the processor, in response to receiving data corresponding to one of the plurality of determination items, the range information and the model information separately from the memory, each of the range information and the model information corresponding to the determination item of the received data,
determining, by the processor, based on the separately obtained range information and the model information, whether a value included in the received data is normal value or abnormal value, and
outputting, by the processor, result of the determination.

5. The method according to claim 4, further comprising
determining, by the processor, when the value of the received data is included in one of the first range and the second range, whether the value included in the received data is normal value or abnormal value based on corresponding determination model among the first, second, and third model indicated by the model information.

6. The method according to claim 4, further comprising
determining, by the processor, when the value of the received data is not included in any of the first range or the second range, whether the value included in the received data is normal value or abnormal value by comparing the value with one of the first value and the second value.

7. A non-transitory computer readable medium having stored therein a program that causes a computer to execute a process, the process comprising:
storing, in a memory, predetermined value independently for each of a plurality of determination items, the predetermined value indicating boundary value to distinguish normal value and abnormal value; and
storing, for each of the determination items, range information indicating first range and second range in the memory in association with corresponding determination item, the first range being independently set for each of the plurality of determination items and variably set for a range between the predetermined value and a first value which is identified as the normal value in comparison with the predetermined value, the second range being independently set for each of the plurality of determination items and variably set for a range between the predetermined value and a second value which is identified as the abnormal value in comparison with the predetermined value,
storing, for each of the determination items, model information that indicates which one of a first model, a second model, and a third model is to be applied to the corresponding determination item in the memory in association with corresponding determination item, the first model being used to determine a value included in the first range as the normal value and to determine a value included in the second range as the abnormal value, the second model being used to determine a value included in the first range as the normal value and to determine a value included in the second range as the normal value, the third model being used to determine a value included in the first range as the abnormal value and to determine a value included in the second range as the abnormal value, such that information used to determine the normal value and the abnormal value is able to be optimized for each of the plurality of determination items by separately storing the range information and the model information in the memory,
obtaining, in response to receiving data corresponding to one of the plurality of determination items, the range information and the model information separately from the memory, each of the range information and the model information corresponding to the determination item of the received data,
determining, based on the separately obtained range information and the model information, whether a value included in the received data is normal value or abnormal value, and
outputting result of the determination.

8. The non-transitory computer readable medium according to claim 7, wherein the process further comprising:
determining, when the value of the received data is included in one of the first range and the second range, whether the value included in the received data is normal value or abnormal value based on corresponding determination model among the first, second, and third model indicated by the model information.

9. The non-transitory computer readable medium according to claim 7, wherein the process further comprising:
determining, when the value of the received data is not included in any of the first range or the second range, whether the value included in the received data is normal value or abnormal value by comparing the value with one of the first value and the second value.
